(19) United States
(12) Patent Application Publication
(10) Pub. No.: US 2011/0052740 A1
(43) Pub. Date: Mar. 3, 2011

(54) Skin Circulation-Improving Agent And Skin Temperature-Elevating Agent

(75) Inventors: Keiko Mori, Kanagawa (JP); Masako Katsuyama, Kanagawa (JP); Yoko Gozu, Kanagawa (JP); Shinichiro Haze, Kanagawa (JP); Atsushi Jouichi, Kanagawa (JP); Yushi Terajima, Kanagawa (JP)

(73) Assignee: SHISEIDO COMPANY, LTD., Chuo-ku, Tokyo (JP)

(21) Appl. No.: 12/935,005

(22) PCT Filed: Mar. 26, 2009
(86) PCT No.: PCT/JP2009/001363

§ 371 Date: Sep. 28, 2010

(30) Foreign Application Priority Data
Mar. 28, 2008 (JP) 2008-085350

(51) Int. Cl. A61K 36/73; A61K 36/63; A61K 31/365; A61K 31/215; A61P 17/00

(52) U.S. Cl. 424/765; 424/725; 514/450; 514/529

(57) ABSTRACT

Skin blood circulation is improved, skin temperature is increased, or shoulder stiffness or sensitivity to cold is prevented or improved by using, as an active ingredient, at least one of the members selected from the group consisting of an Arabian jasmine (matsurika, jasmin sambac) extract, a damask rose (Rosa damascena) steam distillate, 3-methyloctano-4-lactone, and bornyl acetate.

### TECHNICAL FIELD

This invention relates to a skin circulation-improving agent for increasing a rate of skin blood circulation and a skin temperature-elevating agent for increasing skin temperature.

### BACKGROUND ART

In the modern society, there has been an increase in the number of persons who complain of symptoms of sensitivity of the limbs to cold, numbness of the limbs, shoulder stiffness, swelling, and the like, due to various factors, such as lack of exercise, long-period labor in a specific posture at jobsites, air conditioning, and autonomic ataxia due to stress. It is thought that the symptoms described above are caused to occur primarily by a bad condition of blood circulation.

If the circulation of the blood becomes poor due to badness or interruption in blood circulation, or the like, the warm blood does not sufficiently circulate to the periphery. Also, supply of oxygen and nutrients from the blood becomes stagnant. Moreover, waste materials and water are accumulated.

Also, humans, who are homoiothermic animals, are provided with a body temperature regulating function for keeping the body temperature at a specific value. The autonomic nervous system and the endocrine system are regulated by a command given from a body temperature regulation center, which is located at the hypothalamus of the brain and has perceived a change (stimulus) of the ambient temperature, and the body temperature is thereby kept at the specific value. However, if balance of the autonomic nerve is lost due to a mental or physical stress, or the like, the body temperature regulating function does not work well, and the body cannot appropriately cope with the cold. Also, the sympathetic nerve becomes predominant, contraction of blood vessels and tension of the muscles are sustained, and the badness in blood circulation thus arises.

Various attempts have been made in order to improve the symptoms accompanying the bad condition of blood circulation described above. For example, in patent literature 1, it is described that a hyssop extract improves blood fluidity and blood circulation, increases skin surface temperature, and can improve sensitivity to cold and shoulder stiffness.

Also, in patent literature 2, it is described that a composition, which contains a cassis condensate and an amino acid or an organic acid, expands blood vessels, increases a peripheral blood circulation rate, and improves shoulder stiffness and sensitivity to cold.

Further, in patent literature 3, it is described that a smell of a chamomile extract improves blood circulation and increases peripheral skin temperature.

However, there are large individual differences in sensitivity and preference with respect to the aforesaid compounds and compositions having been reported heretofore, and sufficiently satisfactory results have not yet been obtained with the aforesaid compounds and compositions. Therefore, there is a strong demand for a novel substance, which can more efficiently and generally achieve improvement in skin blood circulation and increase in skin temperature.

### Patent Literature 1:

Japanese Unexamined Patent Publication No. 2006-8575

### Patent Literature 2:

Japanese Unexamined Patent Publication No. 2004-262878

Patent literature 3:

Japanese Unexamined Patent Publication No. 2005-68069

### DISCLOSURE OF INVENTION

### Problems to be Solved by the Invention

In view of the above circumstances, the object of the present invention is to provide a novel substance, which can safely and efficiently achieve increase in a rate of skin blood circulation and increase in skin temperature and can efficiently improve sensitivity to cold, shoulder stiffness, and the like.

### Means for Solving the Problems

The inventors have found that each of an extract of Arabian jasmine (matsurika, jasminsambac), which is a plant of the Jasminum genus in the Oleaceae family, a steam distillate of damask rose (Rosa damascena), which is a plant of the rose Turkish Rosaceae family, 3-methyloctano-4-lactone, and bornyl acetate increases the rate of the skin blood circulation and increases the skin temperature. The present invention is based upon the findings described above.

Each of a skin circulation-improving agent and a skin temperature-elevating agent in accordance with the present invention is characterized by containing, as an active ingredient, at least one of the members selected from the group consisting of an Arabian jasmine (matsurika, jasmin sambac) extract, a damask rose (Rosa damascena) steam distillate, 3-methyloctano-4-lactone, and bornyl acetate. The inventors have confirmed that each of the above-enumerated compounds has a sympathetic nerve suppressing effect. It is presumed that, in cases where the autonomic nerve is sedately adjusted to regulate such that the working of the parasympathetic nerve becomes relatively predominant over the working of the sympathetic nerve, the blood circulation is improved through expansion of blood vessels and relief of the tension of the muscles, and the skin temperature increases by virtue of the warm blood circulating to the periphery through the good blood circulation.

Each of an agent for preventing or improving shoulder stiffness and an agent for preventing or improving sensitivity to cold in accordance with the present invention is characterized by containing, as an active ingredient, at least one of the members selected from the group consisting of an Arabian jasmine (matsurika, jasmin sambac) extract, a damask rose (Rosa damascena) steam distillate, 3-methyloctano-4-lactone, and bornyl acetate. In cases where the skin blood circulation is improved, and the skin temperature is increased, the warm blood circulates to the periphery, and metabolism is enhanced. Also, the supply of oxygen and nutrients from the blood and excretion of waste materials are enhanced, and the symptoms of the sensitivity to cold and the shoulder stiffness can be alleviated.

### Effects of the Invention

Each of the skin circulation-improving agent, the skin temperature-elevating agent, and the like, in accordance with the present invention does not have a side effect, can increase the peripheral blood circulation rate or the skin temperature without being influenced by the sensitivities and the preferences of the individuals, and can safely and efficiently improve various symptoms due to a bad condition of blood circulation, such as the sensitivity to cold and the shoulder stiffness.

### BEST MODE FOR CARRYING OUT THE INVENTION

Each of the skin circulation-improving agent, the skin temperature-elevating agent, the agent for preventing or improving shoulder stiffness, and the agent for preventing or improving sensitivity to cold (hereinbelow referred to as the skin circulation-improving agent or the like) in accordance with the present invention contains, as the active ingredient, at least one of the members selected from the group consisting of the Arabian jasmine (matsurika, jasmin sambac) extract, the damask rose (Rosa damascena) steam distillate, 3-methyloctano-4-lactone, and bornyl acetate.

The term “skin circulation improving” as used herein means that the rate of the blood circulation through the skin blood vessels is increased. For example, the improvement in skin circulation can be evaluated by fitting a probe to a finger, a toe, or the like, and measuring a change in blood circulation rate by use of a laser Doppler flow-meter (ADVANCE LASER FLOW-METER ALF21), or the like.

Also, the term “skin temperature elevating” as used herein means that the skin temperature is increased. For example, the elevation of the skin temperature can be evaluated by measuring a change in skin temperature of a cheek, a finger, a toe, or the like, by use of an 8ch body temperature logger (supplied by Nikkiso-Therm Co., Ltd.), or the like.

Further, the term “preventing or improving” as used herein means that the occurrence of the symptoms is prevented, or that the symptoms are alleviated or relieved.

The Arabian jasmine (matsurika, Jasmin sambac) is the plant of the Jasminum genus in the Oleaceae family. Whole grass, leaves, bark, branches, fruits, roots, or the like, of the Arabian jasmine can be used as such or after being pulverized. Particularly, flowers should preferably be used. The Arabian jasmine extract embraces in its scope an extract obtained from processing, wherein the extract part of the plant is subjected as such to the extraction, or processing, wherein the extract part of the plant is subjected to cutting into appropriate sizes or pulverization after being dried and is then subjected to the extraction, and a more active fraction (ingredient) obtained by further subjecting the aforesaid extract to separation and purification. For the extraction, it is possible to employ a solvent extraction technique, wherein the extract part of the plant is impregnated with a solvent at the room temperature or in a heated state, or wherein the extraction is performed by use of an extraction appliance, such as a Soxhlet's extractor, an extraction technique using a distillation technique, such as steam distillation, a super critical extraction technique, wherein the extraction is performed with a carbonic acid gas being set in a super critical state, a press technique, wherein an extract is obtained through pressing, or the like. Particularly, the extract obtained from the solvent extraction can be used preferably.

As the extraction solvent used for the solvent extraction, a polar solvent or a non-polar solvent can be used, and a mixture thereof can also be used. Examples of the extraction solvents include water; alcohols, such as methanol, ethanol, propanol, and butanol; polyhydric alcohols, such as ethylene glycol, propylene glycol, and butylene glycol; ketones, such as acetone and methyl ethyl ketone; esters, such as methyl acetate and ethyl acetate; chain and cyclic ethers, such as tetrahydrofuran and diethyl ether; polyethers, such as polyethylene glycol; halogenated hydrocarbons, such as dichloromethane, chloroform, and carbon tetrachloride; hydrocarbons, such as hexane, cyclohexane, and petroleum ether; aromatic hydrocarbons, such as benzene and toluene; pyridines; super critical carbon dioxide; fats and oils; wax; and other kinds of oils . Each of the above-enumerated solvents can be used alone, or at least two of the above-enumerated solvents can be used in combination. It is also possible to iterate extraction processes by using different kinds of the solvents. Of the above-enumerated solvents, it is preferable to use ethanol, dipropylene glycol, propylene glycol, and butylene glycol. The extraction can be performed, for example, by using 1 part to 50 parts by mass of the solvent with respect to 1 part by mass of the plant, and carrying out dipping or heat reflux at a temperature falling within the range of 3° C. to 100° C. for a period of time falling within the range of several hours to several weeks.

The damask rose (Rosa damascena) is the plant of the rose Turkish Rosaceae family. Whole grass, flowers, leaves, bark, branches, fruits, roots, or the like, of the damask rose can be used as such or after being pulverized. Particularly, flowers should preferably be used. The term “steam distillate” as used herein means the essential oil obtained with the steam distillation. The damask rose steam distillate used in the present invention can be prepared with processing, wherein the extract part of the plant is subjected as such to the ordinary steam distillation, or processing, wherein the extract part of the plant is subjected to cutting into appropriate sizes or pulverization after being dried and is then subjected to the ordinary steam distillation. The damask rose steam distillate also embraces in its scope a more active fraction (ingredient) obtained by further subjecting the aforesaid steam distillate to the separation and the purification. For example, the damask rose steam distillate can be prepared by blowing steam to the flowers or the dried flowers of the damask rose, collecting the resulting volatilized substance with cooling, and separating and recovering the oily part (the essential oil).

Each of the Arabian jasmine extract and the damask rose steam distillate may further be subjected to the separation and the purification. Examples of means for the separation and the purification include activated carbon treatment, liquid-liquid distribution, column chromatography, liquid chromatography, gel filtration, and precision distillation.

Each of 3-methyloctano-4-lactone and bornyl acetate used in the present invention can be synthesized by use of a known synthetic technique. Alternatively, a commercially available product may be used.

In the present invention, one of the members selected from the group consisting of the Arabian jasmine extract, the damask rose steam distillate, 3-methyloctano-4-lactone, and bornyl acetate may be used alone. Alternatively, at least two of the members selected from the group described above may be used in combination. In so far as the skin circulation-improving agent or the like in accordance with the present invention contains at least one of the aforesaid substances as the active ingredient and can accomplish the effects of the present invention, the form of the skin circulation-improving agent or the like is not limited particularly, and the skin circulation-improving agent or the like can be used in an arbitrary form, such as a liquid, a paste, a gel, or a solid. Also, the skin circulation-improving agent or the like in accordance with the present invention can contain other arbitrary ingredients, such as carriers, diluents, and auxiliaries, within a range such that the effects of the present invention are not obstructed.

The skin circulation-improving agent or the like in accordance with the present invention can be applied to arbitrary use applications, such as pharmaceutical preparations, quasi-drugs, cosmetic preparations, foods, and drinks. Also, since the active ingredient described above can exhibit the effects through vaporization, the skin circulation-improving agent or the like in accordance with the present invention can also be applied to clothes, daily-use goods, and the like. The object, in which the skin circulation-improving agent or the like in accordance with the present invention is contained, may be an arbitrary object, which can contain the skin circulation-improving agent or the like in a form such that the active ingredient described above can be vaporized and inhaled as vapor. The object is not limited by the preparation form and the product form. In accordance with the kind of the object, the skin circulation-improving agent or the like in accordance with the present invention may further contain arbitrary constituent elements, which are ordinarily contained in the object, besides the active ingredient described above. Furthermore, other arbitrary active drugs and the skin circulation-improving agent or the like in accordance with the present invention may be used together with each other.

Though it is not limited, examples of the product forms containing the skin circulation-improving agent or the like in accordance with the present invention include cosmetic preparations, pharmaceutical preparations, quasi-drugs, foods, and drinks. Also, examples of the preparation forms include liquids, powders, granules, aerosols, solids, and gels.

Though it is not limited, examples of the cosmetic preparations, which are one of particularly preferable embodiments, include perfumes, eau de toilette, eau de Cologne, creams, milky lotions, skin lotions, foundations, face powders, lipsticks, soaps, shampoos and rinses, body shampoos, body rinses, body powders, and bath preparations.

Also, the skin circulation-improving agent or the like in accordance with the present invention can be contained in arbitrary daily-use goods, such as aromatic products, deodorants, aroma candles, incense, writing materials, purses, bags, and shoes; and arbitrary clothes, such as underwear, dresses, hats and caps, stockings, and socks. The skin circulation-improving agent or the like in accordance with the present invention may be added to the materials for the daily-use goods and the clothes described above or the products of the daily-use goods and the clothes described above.

The skin circulation-improving agent or the like in accordance with the present invention can be used in various embodiments exemplified above, but the embodiments of the skin circulation-improving agent or the like in accordance with the present invention are not limited to those exemplified above. In so far as the effects of the present invention can be accomplished, the skin circulation-improving agent or the like in accordance with the present invention can be used in arbitrary embodiments.

### Examples

The present invention will further be illustrated by the following non-limitative examples. As the Arabian jasmine solvent extract and the damask rose steam distillate, those commercially available from Robertet were used. As 3-methyloctano-4-lactone, the compound commercially available from Soda Aromatic Co., Ltd. was used. Also, as bornyl acetate, the compound commercially available from Takasago International Corporation was used.

### Test Example 1

### Study of Effect on Skin Temperature

The skin temperature of fingers was measured by use of 8ch Body Temperature Logger (supplied by Nikkiso-Therm Co., Ltd.), and a change in skin temperature due to smelling of a test substance was investigated. The effect on the peripheral skin temperature was thus studied. A solvent extract of a common jasmine (Jasminum officinale) belonging to the Jasminum genus in the Oleaceae family was used as a control, and the evaluation was made in the same manner.

A woman in her twenties was employed as a subject, and the test was made in a constant temperature and humidity room at a temperature of 25° C. and a humidity of 50%.

The subject was seated on an easy chair and allowed to take a rest for 30 minutes. Thereafter, body temperature sensors were fitted to inner surface sides of third fingers of both hands of the subject and to two sites of the chest of the subject, and a change in skin temperature at each site with the passage of time was recorded. Since it had been found that little change arose in skin temperature at the chest, the skin temperatures at the chest were employed as reference values. The change in peripheral skin temperature was evaluated by use of a value (T value) obtained by subtracting a mean value of the skin temperatures at the two sites of the chest from the mean value of the skin temperatures at the two fingertip sites.

One sequence of measurement tests was composed of (A) a rest for three minutes, (B) attaching of an odorless cotton piece under the nose for three minutes, (C) a rest for three minutes, (D) attaching of a test sample-impregnated cotton piece under the nose for three minutes, (E) a rest for three minutes, (F) attaching of an odorless cotton piece under the nose for three minutes, and (G) a rest for three minutes. The skin temperature was measured successively during the one sequence, and the T values were calculated.

The T values obtained at the times of (B) and (F), at each of which the odorless cotton piece was attached, were taken as the controls and were compared with the T value obtained at the time of (D), at which the vapor of the test sample was inhaled. The effect of each substance on the skin temperature was thus investigated. Specifically, the formula shown below was utilized.

Skin temperature change (° C.)=T value (D)−(mean value of T value (B) and T value (F))

The results are shown in FIG. 1. It was indicated that, in cases where the subject was allowed to take the smell of the Arabian jasmine extract, the damask rose steam distillate, 3-methyloctano-4-lactone, or bornyl acetate with the nose, the skin temperatures of the fingers of the subject were increased, and that each of these substances thus had the skin temperature increasing effect.

Also, though it is not illustrated by data, in cases where the subject was allowed to take the smell of the Arabian jasmine extract, the damask rose steam distillate, 3-methyloctano-4-lactone, or bornyl acetate with the nose, the skin temperatures of the toes and the cheeks of the subject could be increased.

### Test Example 2

### Study of Effect on Skin Blood Circulation

The skin blood circulation rate of fingers was measured by use of the laser Doppler flow-meter (ADVANCE LASER FLOW-METER ALF21), and the effect of the Arabian jasmine extract on the skin blood circulation was evaluated.

Six persons (men and women) were employed as subjects, and the test was made in a constant temperature and humidity room having been adjusted at a temperature of 22° C. and a humidity of 45%. Each of the subjects was seated on an easy chair and allowed to take a rest for 30 minutes. Thereafter, a probe of the flow-meter was fitted to the inner surface side of the third finger of the right hand of the subject, and a change in blood circulation rate with the passage of time was recorded. One sequence of measurement tests was composed of (A) a rest for three minutes, (B) attaching of an odorless cotton piece under the nose for three minutes, (C) a rest for three minutes, (D) attaching of a test sample-impregnated cotton piece under the nose for three minutes, (E) a rest for three minutes, (F) attaching of an odorless cotton piece under the nose for three minutes, and (G) a rest for three minutes. The blood circulation rate was measured successively during the one sequence.

The blood circulation rates obtained at the times of (B) and (F), at each of which the odorless cotton piece was attached, were taken as the controls and were compared with the blood circulation rate obtained at the time of (D), at which the vapor of the test sample was inhaled. The effect of each substance on the skin blood circulation rate was thus investigated. Specifically, the formula shown below was utilized.

Blood circulation rate change (ml/min/100 g)=blood circulation rate (D)−(mean value of blood circulation rate (B) and blood circulation rate (F))

The results are shown in FIG. 2. It was indicated that, in cases where each of the subjects was allowed to take the smell of the Arabian jasmine extract with the nose, the skin blood circulation rate was increased, and that the Arabian jasmine extract thus had the skin blood circulation improving effect.

### Test Example 3

### Study of Effects on Symptoms of Sensitivity to Cold and Shoulder Stiffness

As one of causes of the sensitivity to cold, the shoulder stiffness, and the like, the decreasing in blood circulation is considered. Therefore, the improvement effects of the Arabian jasmine extract on the symptoms of the sensitivity to cold and the shoulder stiffness were investigated.

Four persons (men and women) aware of the sensitivity to cold were employed as the subjects, and the test was made in a constant temperature and humidity room having been adjusted at a temperature of 22° C. and a humidity of 45%. Each of the subjects was seated on an easy chair and allowed to take a rest for 30 minutes. Thereafter, an odorless cotton piece was attached under the nose of the subject. After a period of time of three minutes had elapsed, each of the extents of the sensitivity to cold and the shoulder stiffness was evaluated subjectively on a five-point scale. Thereafter, a cotton piece impregnated with the Arabian jasmine extract was attached under the nose, and the vapor of the extract was inhaled with natural breathing for three minutes. Thereafter, each of the extents of the sensitivity to cold and the shoulder stiffness was again evaluated subjectively on the five-point scale.

The results are shown in FIG. 3. It was indicated that, in cases where each of the subjects was allowed to take the smell of the Arabian jasmine extract with the nose, the results of the warming of the hands, feet, and the entire body were obtained as an actual feeling and that, in such cases, the symptoms of the shoulder stiffness were improved.

Application examples of typical product forms, such as various compositions, daily-use goods, and clothes, containing the skin circulation-improving agent, the skin temperature-elevating agent, the agent for preventing or improving shoulder stiffness, or the agent for preventing or improving sensitivity to cold in accordance with the present invention will be described hereinbelow. In each of the application examples, the containing quantity is expressed in terms of % by mass with respect to the total quantity of the product. As the skin circulation-improving agent or the like in accordance with the present invention, one of the members selected from the group consisting of the Arabian jasmine extract, the damask rose steam distillate, 3-methyloctano-4-lactone, and bornyl acetate was contained alone, or at least two of the members selected from the group described above were contained in combination.

### Application Example 1

Skin lotion

| | | |
|---|---|---|
| (1) | Glycerol | 2.0 |
| (2) | Dipropylene glycol | 2.0 |
| (3) | PEG-60 hydrogenated castor oil | 0.3 |
| (4) | Trimethylglycine | 0.1 |
| (5) | Preservative | Proper quantity |
| (6) | Chelating agent | Proper quantity |
| (7) | Dye | Proper quantity |
| (8) | Skin circulation-improving agent | 0.05 |
| | (Arabian jasmine (matsurika, | |
| | jasmin sambac) extract) | |
| (9) | Purified water | Balance |

### Application Example 2

Skin lotion

| | | |
|---|---|---|
| (1) | Alcohol | 30.0 |
| (2) | Butylene glycol | 4.0 |
| (3) | Glycerol | 2.0 |
| (4) | PPG-13 decyltetradeth-24 | 0.3 |
| (5) | Octylmethoxy cinnamate | 0.1 |
| (6) | Menthol | 0.2 |
| (7) | Tocopheryl acetate | 0.1 |
| (8) | Chelating agent | Proper quantity |
| (9) | Dye | Proper quantity |
| (10) | Skin circulation-improving agent | 0.01 |
| | (bornyl acetate) | |
| (11) | Purified water | Balance |

### Application Example 3

Milky lotion

| | | |
|---|---|---|
| (1) | Stearic acid | 2.0 |
| (2) | Cetyl alcohol | 1.5 |
| (3) | Vaseline | 4.0 |
| (4) | Squalane | 5.0 |
| (5) | Glycerol tri-2-ethylhexanoic | 2.0 |
| | acid ester | |
| (6) | Sorbitan monooleic acid ester | 2.0 |
| (7) | Dipropylene glycol | 5.0 |
| (8) | PEG1500 | 0.3 |
| (9) | Triethanolamine | 0.1 |
| (10) | Preservative | Proper quantity |
| (11) | Skin circulation-improving agent | 0.2 |
| | (3-methyloctano-4-lactone) | |
| (12) | Purified water | Balance |

### Application Example 4

Milky lotion

| | | |
|---|---|---|
| (1) | Ethyl alcohol | 10.0 |
| (2) | Cyclomethicone | 0.1 |
| (3) | Butylene glycol | 5.0 |
| (4) | Dimethicone | 3.0 |
| (5) | Glycene | 0.1 |
| (6) | Menthol | 1.0 |
| (7) | Trimethylsiloxysilicic acid | 0.1 |
| (8) | Caffeine | 1.0 |
| (9) | Trimethylglycine | 1.0 |
| (10) | Xanthan gum | 0.001 |
| (11) | Hydroxyethyl cellulose | 0.1 |
| (12) | Soybean fermentation extract | 1.0 |
| (13) | Lauryl betaine | 0.5 |
| (14) | Carbomer | 0.2 |
| (15) | Chelating agent | Proper quantity |
| (16) | Paraben | Proper quantity |
| (17) | Benzoic acid | Proper quantity |
| (18) | Skin temperature-elevating agent | 0.1 |
| | (bornyl acetate) | |
| (19) | Iron oxide | Proper quantity |
| (20) | Potassium hydroxide | 0.05 |
| (21) | Dicalcium glycyrrhizinate | 0.01 |
| (22) | Pyridoxine hydrochloride | 0.01 |
| (23) | Ascorbic acid glucoside | 0.01 |
| (24) | Arbutin | 3.0 |
| (25) | Extract of Saxifraga stolonifera | 0.1 |
| | Meerb. | |
| (26) | Water | Balance |

### Application Example 5

Milky lotion

| | | |
|---|---|---|
| (1) | Butylene glycol | 4.0 |
| (2) | Propylene glycol | 4.0 |
| (3) | Carbomer | 0.2 |
| (4) | Potassium hydroxide | 0.2 |
| (5) | Behenic acid | 0.5 |
| (6) | Stearic acid | 0.5 |
| (7) | Isostearic acid | 0.5 |
| (8) | Glyceryl stearate | 1.0 |
| (9) | Glyceryl isostearate | 1.0 |
| (10) | Behenyl alcohol | 0.5 |
| (11) | Squalane | 5.0 |
| (12) | Trioctanoin | 3.0 |
| (13) | Phenyl trimethicone | 2.0 |
| (14) | Batyl alcohol | 0.5 |
| (15) | Dicalcium glycyrrhizinate | 0.01 |
| (16) | Preservative | Proper quantity |
| (17) | Chelating agent | Proper quantity |
| (18) | Pigment | Proper quantity |
| (19) | Skin temperature-elevating agent | 0.1 |
| | (damask rose (Rosa damascena) | |
| | steam distillate) | |
| (20) | Skin temperature-elevating agent | 0.05 |
| | (Arabian jasmine (matsurika, | |
| | jasmin sambac) extract) | |
| (21) | Purified water | Balance |

### Application Example 6

Milky lotion

| | | |
|---|---|---|
| (1) | Glycerol | 3.0 |
| (2) | Xylitol | 2.0 |
| (3) | Carbomer | 0.1 |
| (4) | Potassium hydroxide | 0.1 |
| (5) | Glyceryl isostearate | 1.0 |
| (6) | Glyceryl stearate | 0.5 |
| (7) | Behenyl alcohol | 1.0 |
| (8) | Batyl alcohol | 1.0 |
| (9) | Hydrogenated palm oil | 2.0 |
| (10) | Vaseline | 1.0 |
| (11) | Squalane | 5.0 |
| (12) | Erythrityl octanoate | 3.0 |
| (13) | Cyclomethicone | 1.0 |
| (14) | Preservative | Proper quantity |
| (15) | Chelating agent | Proper quantity |
| (16) | Skin circulation-improving agent | 0.2 |
| | (damask rose (Rosa damascena) | |
| | steam distillate) | |
| (17) | Purified water | Balance |
| (18) | Tranexamic acid | 1.0 |
| (19) | Pantothenyl ethyl ether | 0.5 |
| (20) | Nicotinic acid amide | 0.1 |
| (21) | Trehalose | 0.1 |
| (22) | Rosemary extract | 0.1 |
| (23) | Vitamin A | 0.1 |
| (24) | Ascorbic acid glycoside | 0.001 |
| (25) | Raspberry extract | 1.0 |
| (26) | Scutellaria Root extract | 0.001 |
| (27) | Phellodendron Bark extract | 0.01 |

### Application Example 7

Milky lotion

| | | |
|---|---|---|
| (1) | Ethanol | 2.0 |
| (2) | Cyclomethicone | 10.0 |
| (3) | Glycerol | 5.0 |
| (4) | Dibutylene glycol | 1.0 |
| (5) | Dimethicone | 1.0 |
| (6) | Corn starch | 4.0 |
| (7) | Mineral oil | 2.0 |
| (8) | Trimethylsiloxysilicic acid | 5.0 |
| (9) | Polyethylene glycol | 3.0 |
| (10) | Menthyl lactate | 0.1 |
| (11) | PEG-60 hydrogenated castor oil | 1.0 |
| (12) | Aminopropyl dimethicone | 1.0 |
| (13) | Xanthan gum | 0.01 |
| (14) | Tocopheryl acetate | 0.01 |
| (15) | Caffeine | 0.1 |
| (16) | Sodium hyaluronate | 0.1 |
| (17) | Soybean fermentation extract | 0.01 |
| (18) | Hamamelis extract | 0.01 |
| (19) | Extract of Houttuynia cordata | 0.01 |
| (20) | Carbomer | 0.3 |
| (21) | Acrylic acid-alkyl matacrylate | 0.2 |
| | copolymer | |
| (22) | EDTA | Proper quantity |
| (23) | Preservative | Proper quantity |
| (24) | Skin temperature-elevating agent | 0.3 |
| | (bornyl acetate) | |
| (25) | Pigment | Proper quantity |
| (26) | Potassium hydroxide | 0.15 |
| (27) | Aminomethylpropanol | 0.05 |
| (28) | Water | Balance |

### Application Example 8

Milky lotion

| | | |
|---|---|---|
| (1) | Ethanol | 15.0 |
| (2) | Cyclomethicone | 6.0 |
| (3) | Butylene glycol | 0.5 |
| (4) | Dimethicone | 1.0 |
| (5) | Glycerol | 1.0 |
| (6) | Polyethylene glycol | 1.0 |
| (7) | Menthyl lactate | 1.0 |
| (8) | Menthol | 0.1 |
| (9) | Trimethylsiloxysilicic acid | 1.0 |
| (10) | Caffeine | 0.5 |
| (11) | Trimethyleneglycine | 0.1 |
| (12) | Xanthan gum | 0.1 |
| (13) | Hydroxyethyl cellulose | 0.1 |
| (14) | Soybean fermentation extract | 0.01 |
| (15) | Tocopheryl acetate | 0.05 |
| (16) | Lauryl betaine | 0.01 |
| (17) | Brown algae extract | 0.01 |
| (18) | Extract of Houttuynia cordata | 0.01 |
| (19) | Red algae extract | 0.01 |
| (20) | Green algae extract | 0.01 |
| (21) | Cellulose powder | 1.0 |
| (22) | PEG-60 glyceryl isostearate | 1.0 |
| (23) | Isostearic acid | 1.0 |
| (24) | Carbomer | 0.1 |
| (25) | Acrylic acid-alkyl methacrylate | 0.1 |
| | copolymer | |
| (26) | EDTA | 0.1 |
| (27) | Sodium metaphosphate | 0.1 |
| (28) | Phenoxy ethanol | 0.2 |
| (29) | Paraben | 0.2 |
| (30) | Skin temperature-elevating agent | 0.20 |
| | (3-methyloctano-4-lactone) | |
| (31) | Skin temperature-elevating agent | 0.15 |
| | (bornyl acetate) | |
| (32) | Iron oxide (red) | 0.02 |
| (33) | Menthyl glyceryl ether | 0.01 |
| (34) | Water | Balance |

### Application Example 9

Cream

| | | |
|---|---|---|
| (1) | Glycerol | 10.0 |
| (2) | Butylene glycol | 5.0 |
| (3) | Carbomer | 0.1 |
| (4) | Potassium hydroxide | 0.2 |
| (5) | Stearic acid | 2.0 |
| (6) | Glyceryl starate | 2.0 |
| (7) | Glyceryl isostearate | 2.0 |
| (8) | Vaseline | 5.0 |
| (9) | Preservative | Proper quantity |
| (10) | Antioxidant | Proper quantity |
| (11) | Skin circulation-improving agent | 0.3 |
| | (damask rose (Rosa damascena) | |
| | steam distillate) | |
| (12) | Purified water | Balance |
| (13) | Chelating agent | Proper quantity |
| (14) | Pigment | Proper quantity |
| (15) | Stearyl alcohol | 2.0 |
| (16) | Behenyl alcohol | 2.0 |
| (17) | Hydrogenated palm oil | 2.0 |
| (18) | Squalane | 10.0 |
| (19) | Potassium 4-methoxysalicylate | 3.0 |

### Application Example 10

Cream

| | | |
|---|---|---|
| (1) | Glycerol | 3.0 |
| (2) | Dipropylene glycol | 7.0 |
| (3) | Polyethylene glycol | 3.0 |
| (4) | Glyceryl stearate | 3.0 |
| (5) | Glyceryl isostearate | 2.0 |
| (6) | Stearyl alcohol | 2.0 |
| (7) | Behenyl alcohol | 2.0 |
| (8) | Liquid paraffin | 7.0 |
| (9) | Cyclomethicone | 3.0 |
| (10) | Dimethicone | 1.0 |
| (11) | Octylmethoxy cinnamate | 0.1 |
| (12) | Sodium hyaluronate | 0.05 |
| (13) | Preservative | Proper quantity |
| (14) | Antioxidant | Proper quantity |
| (15) | Skin circulation-improving agent | 0.3 |
| | (Arabian jasmine (matsurika, | |
| | Jasmin sambac) extract) | |
| (16) | Skin circulation-improving agent | 0.1 |
| | (3-methyloctano-4-lactone) | |
| (17) | Purified water | Balance |
| (18) | Chelating agent | Proper quantity |
| (19) | Pigment | Proper quantity |

### Application Example 11

Gel

| | | |
|---|---|---|
| (1) | Ethyl alcohol | 10.0 |
| (2) | Glycerol | 5.0 |
| (3) | Butylene glycol | 5.0 |
| (4) | Carbomer | 0.5 |
| (5) | Aminomethylpropanol | 0.3 |
| (6) | PEG-60 hydrogenated castor oil | 0.3 |
| (7) | Menthol | 0.02 |
| (8) | Preservative | Proper quantity |
| (9) | Chelating agent | Proper quantity |
| (10) | Skin temperature-elevating agent | 0.05 |
| | (damask rose (Rosa damascena) | |
| | steam distillate) | |
| (11) | Skin temperature-elevating agent | 0.1 |
| | (bornyl acetate) | |
| (12) | Purified water | Balance |

### Application Example 12

Aerosol

| | | |
|---|---|---|
| (1) | Glycerol | 2.0 |
| (2) | Dipropylene glycol | 2.0 |
| (3) | PEG-60 hydrogenated castor oil | 0.3 |
| (4) | HPPCD | 1.0 |
| (5) | Preservative | Proper quantity |
| (6) | Chelating agent | Proper quantity |
| (7) | Dye | Proper quantity |
| (8) | Skin circulation-improving agent | 0.2 |
| | (Arabian jasmine (matsurika, | |
| | jasmin sambac) extract) | |
| (9) | Purified water | Proper quantity |
| (10) | LPG | Balance |

### Application Example 13

Aerosol

| | | |
|---|---|---|
| (1) | Alcohol | 15.0 |
| (2) | Butylene glycol | 2.0 |
| (3) | Glycerol | 1.0 |
| (4) | PPG-13 decyltetradeth-24 | 0.1 |
| (5) | Silver-carrying zeolite | 1.0 |
| (6) | Chelating agent | Proper quantity |
| (7) | Dye | Proper quantity |
| (8) | Skin circulation-improving agent | 0.15 |
| | (bornyl acetate) | |
| (9) | Purified water | Balance |
| (10) | LPG | 40.0 |

### Application Example 14

Aerosol

| | | |
|---|---|---|
| (1) | Ethanol | 60.0 |
| (2) | Menthyl lactate | 0.1 |
| (3) | Sodium lactate | 0.1 |
| (4) | Tocopheryl acetate | 0.01 |
| (5) | Lactic acid | 0.01 |
| (6) | Caffeine | 0.01 |
| (7) | Fennel extract | 1.0 |
| (8) | Hamamelis extract | 1.0 |
| (9) | Extract of Houttuynia cordata | 1.0 |
| (10) | Dipropylene glycol | 1.0 |
| (11) | Nitrogen gas | 0.9 |
| (12) | Polyoxyethylene polyoxypropylene | 1.0 |
| | decyltetradecyl ether | |
| (13) | Butylene glycol | 2.0 |
| (14) | Tocopherol | 0.05 |
| (15) | Skin temperature-elevating agent | 0.05 |
| | (Arabian jasmine (matsurika, | |
| | jasmin sambac) extract) | |
| (16) | Skin temperature-elevating agent | 0.05 |
| | (damask rose (Rosa damascena) | |
| | steam distillate) | |
| (17) | PEG-60 hydrogenated castor oil | 0.1 |
| (18) | Water | Balance |

### Application Example 15

Shampoo

| | | |
|---|---|---|
| (1) | Lauryl polyexyethylene (3) | 30.0 |
| | sulfuric acid ester sodium salt | |
| | (30% aqueous solution) | |
| (2) | Lauryl sulfuric acid ester | 10.0 |
| | sodium salt (30% aqueous solution) | |
| (3) | Coconut oil fatty acid | 4.0 |
| | diethanolamide | |
| (4) | Glycerol | 1.0 |
| (5) | Preservative | Proper quantity |
| (6) | Skin circulation-improving agent | 0.5 |
| | (bornyl acetate) | |
| (7) | Colorant | Proper quantity |
| (8) | Sequestering agent, pH adjustor | Proper quantity |
| (9) | Purified water | Balance |

### Application Example 16

Rinse

| | | |
|---|---|---|
| (1) | Silicone oil | 3.0 |
| (2) | Liquid paraffin | 1.0 |
| (3) | Cetyl alcohol | 1.5 |
| (4) | Stearyl alcohol | 1.0 |
| (5) | Stearyl trimethyl ammonium chloride | 0.7 |
| (6) | Glycerol | 3.0 |
| (7) | Skin temperature-elevating agent | 0.5 |
| | (damask rose (Rosa damascena) | |
| | steam distillate) | |
| (8) | Colorant, preservative | Proper quantity |
| (9) | Purified water | Balance |

### Application Example 17

Body shampoo

| | | |
|---|---|---|
| (1) | Lauric acid | 2.5 |
| (2) | Myristic acid | 5.0 |
| (3) | Palmitic acid | 2.5 |
| (4) | Oleic acid | 2.5 |
| (5) | Cocoyl diethanolamide | 1.0 |
| (6) | Glycerol | 20.0 |
| (7) | Potassium hydroxide | 3.6 |
| (8) | Dye | Proper quantity |
| (9) | Skin circulation-improving agent | 0.5 |
| | (damask rose (Rosa damascena) | |
| | steam distillate) | |
| (10) | Sequestering agent | Proper quantity |
| (11) | Purified water | Balance |

### Application Example 18

Fragrance

| | | |
|---|---|---|
| (1) | Alcohol | 75.0 |
| (2) | Purified water | Balance |
| (3) | Dipropylene glycol | 5.0 |
| (4) | Skin temperature-elevating agent | 10.0 |
| | (3-methyloctano-4-lactone) | |
| (5) | Antioxidant | 8.0 |
| (6) | Colorant | Proper quantity |
| (7) | UV-absorber | Proper quantity |

The term “fragrance” as used herein means the solution of the essential oil dissolved in an alcohol (e.g., ethyl alcohol) or an aqueous alcohol. The fragrance contains the essential oil in a containing quantity falling within the range of 1% to 99% by mass. The containing ratio of water to the alcohol falls within the range between 50:50 and 0:100. The fragrance can contain solubilizers, softening agents, humectants, thickening agents, bacteriostatic agents, or other materials which are ordinarily used in cosmetic preparations.

### Application Example 19

Room fragrance

| | | |
|---|---|---|
| (1) | Alcohol | 80.0 |
| (2) | Purified water | Balance |
| (3) | Antioxidant | 5.0 |
| (4) | Skin circulation-improving agent | 2.0 |
| | (Arabian jasmine (matsurika, | |
| | jasmin sambac) extract) | |
| (5) | Skin circulation-improving agent | 1.0 |
| | (3-methyloctano-4-lactone) | |
| (6) | 3-Methyl-3-methoxy butanol | 5.0 |
| (7) | Dibenzylidene sorbitol | 5.0 |

### Application Example 20

Incense

| | | |
|---|---|---|
| (1) | Machilus thunbergii powder | 75.5 |
| (2) | Sodium benzoate | 15.5 |
| (3) | Skin circulation-improving agent | 5.0 |
| | (bornyl acetate) | |
| (4) | Eucalyptus oil | 1.0 |
| (5) | Fennel oil | 1.0 |
| (6) | Purified water | Balance |

### Application Example 21

Bath preparation

| | | |
|---|---|---|
| (1) | Sodium sulfate | 45.0 |
| (2) | Sodium hydrogencarbonate | 45.0 |
| (3) | Hyssop oil | 9.0 |
| (4) | Skin temperature-elevating agent | 1.0 |
| | (damask rose (Rosa damascena) | |
| | steam distillate) | |

### Application Example 22

Massage gel

| | | |
|---|---|---|
| (1) | Erythritol | 2.0 |
| (2) | Caffeine | 5.0 |
| (3) | Phellodendron Bark extract | 3.0 |
| (4) | Glycerol | 50.0 |
| (5) | Carboxyvinyl polymer | 0.4 |
| (6) | Polyethylene glycol 400 | 30.0 |
| (7) | Trisodium edetate | 0.1 |
| (8) | Polyoxylene-(10)-methylpolysiloxane | 2.0 |
| | copolymer | |
| (9) | Squalane | 1.0 |
| (10) | Potassium hydroxide | 0.15 |
| (11) | Skin circulation-improving agent | 0.3 |
| | (Arabian jasmine (matsurika, | |
| | jasmin sambac) extract) | |
| (12) | Skin circulation-improving agent | 0.2 |
| | (damask rose (Rosa damascena) | |
| | steam distillate) | |
| (13) | Skin circulation-improving agent | 0.5 |
| | (3-methyloctano-4-lactone) | |

### Application Example 23

Massage cream

| | | |
|---|---|---|
| (1) | Solid paraffin | 5.0 |
| (2) | Beeswax | 10.0 |
| (3) | Vaseline | 15.0 |
| (4) | Liquid paraffin | 41.0 |
| (5) | 1,3-Butylene glycol | 4.0 |
| (6) | Glycerol monostearate | 2.0 |
| (7) | POE (20) sorbitan monolauric | 2.0 |
| | acid ester | |
| (8) | Borax | 0.2 |
| (9) | Caffeine | 2.0 |
| (10) | Preservative | Proper quantity |
| (11) | Antioxidant | Proper quantity |
| (12) | Skin circulation-improving agent | 1.0 |
| | (bornyl acetate) | |
| (13) | Purified water | Balance |

### Application Example 24

### Aromatic Fiber

Micro-capsules (particle diameters: at most 50 μm, ratio of the essential oil occupying the micro-capsules: 50% by weight), in which the skin circulation-improving agent (the damask rose (Rosa damascena) steam distillate) in accordance with the present invention had been encapsulated, were added into a cuprammonium cellulose solution (cellulose concentration: 10% by weight, ammonium concentration: 7% by weight, copper concentration: 3.6% by weight) in a quantity falling within the range of 0.1% to 20% by weight with respect to the weight of the cellulose. The resulting mixture was kneaded and was then spun according to the ordinary wet spinning technique. Scouring and drying were then performed, and an aromatic fiber was thus obtained.

### Application Example 25

Granule

| | | |
|---|---|---|
| (1) | Sucralose | 0.1 |
| (2) | Skin temperature-elevating agent | 0.1 |
| | (damask rose (Rosa damascena) | |
| | steam distillate) | |
| (3) | Flavoring | 5.0 |
| (4) | Excipient (Ceolus) | 10.0 |
| (5) | Maltitol | Balance |

### Application Example 26

Tablet (chewable type)

| | | |
|---|---|---|
| (1) | Inositol | 11.0 |
| (2) | Maltitol | 21.0 |
| (3) | Sucrose | 0.5 |
| (4) | Salmon milt extract (DNA Na) | 0.1 |
| (5) | Yeast extract | 0.1 |
| (6) | Skin circulation-improving agent | 0.1 |
| | (3-methyloctano-4-lactone) | |
| (7) | Flavoring | 5.0 |
| (8) | Excipient | Balance |

### Application Example 27

Tablet

| | | |
|---|---|---|
| (1) | Lubricant (sucrose fatty acid | 1.0 |
| | ester, or the like) | |
| (2) | Aqueous gum arabic solution | 2.0 |
| | (5%) | |
| (3) | Acid taste agent | 1.0 |
| (4) | Colorant | Proper quantity |
| (5) | Skin temperature-elevating agent | 0.1 |
| | (Arabian jasmine (matsurika, | |
| | jasmin sambac) extract) | |
| (6) | Glucide (sugar powder, sorbitol, | Balance |
| | or the like) | |

### Application Example 28

Candy

| | | |
|---|---|---|
| (1) | Sugar | 50.0 |
| (2) | Thick malt syrup | 47.95 |
| (3) | Organic acid | 2.0 |
| (4) | Skin temperature-elevating agent | 0.05 |
| | (bornyl acetate) | |

### Application Example 29

Chewing gum

| | | |
|---|---|---|
| (1) | Sugar | 43.0 |
| (2) | Gum base | 30.95 |
| (3) | Glucose | 10.0 |
| (4) | Thick malt syrup | 16.0 |
| (5) | Skin circulation-improving agent | 0.02 |
| | (damask rose (Rosa damascena) | |
| | steam distillate) | |
| (6) | Skin circulation-improving agent | 0.03 |
| | (3-methyloctano-4-lactone) | |

By use tests having been made in typical embodiments of the corresponding product forms, it was confirmed that each of the various kinds of the compositions, the daily-use goods, the clothes, and the like, obtained in the application examples described above could improve the shoulder stiffness or the sensitivity to cold.

### BRIEF DESCRIPTION OF DRAWINGS

FIG. 1 is a graph showing an effect on skin temperature,

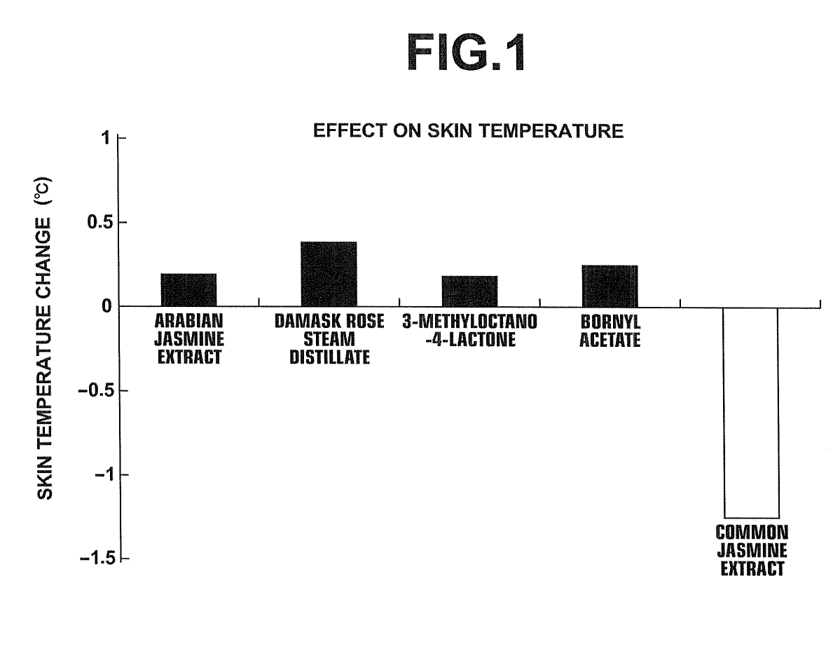

FIG. 2 is a graph showing an effect on skin blood circulation, and

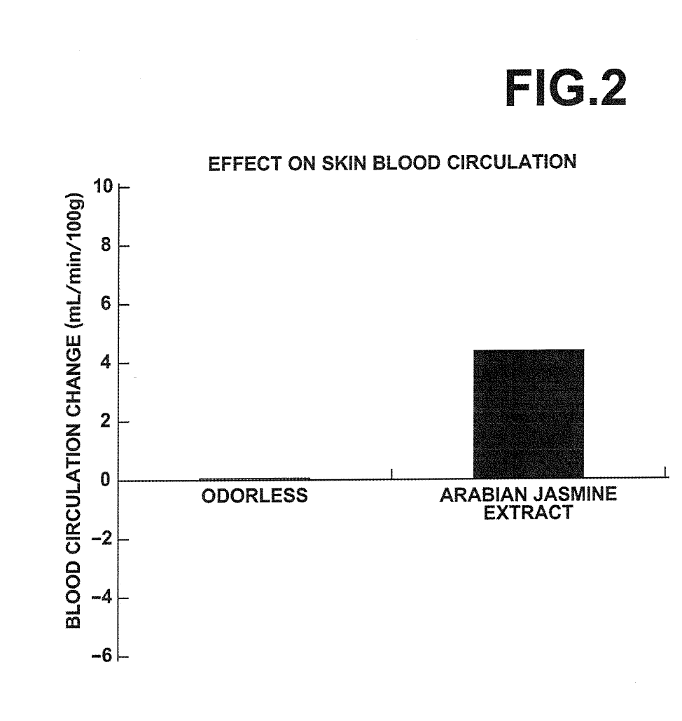

FIG. 3 is a graph showing effects on symptoms of sensitivity to cold and shoulder stiffness.

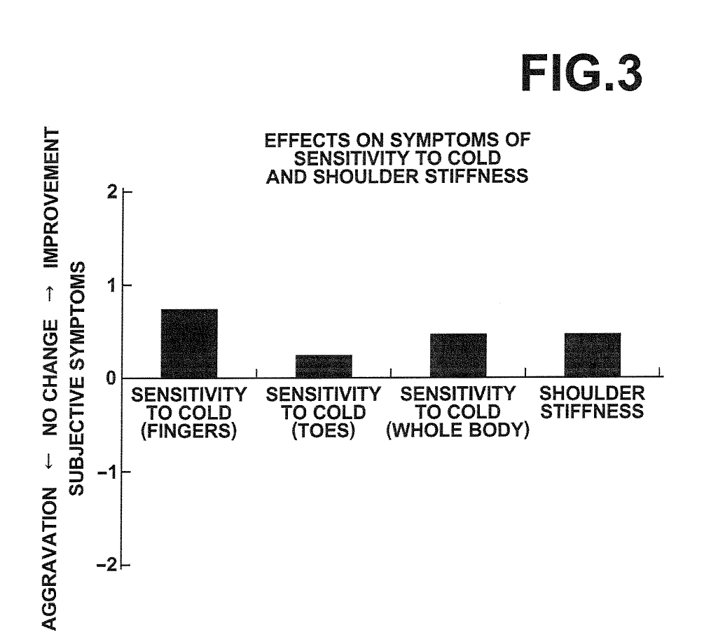

1-4. (canceled)

5. A method of improving skin blood circulation, comprising applying a composition comprising an active ingredient to a subject, wherein the active ingredient comprises at least one of the members selected from the group consisting of an Arabian jasmine (matsurika, jasmin sambac) extract, a damask rose (Rosa damascena) steam distillate, 3-methyloctano-4-lactone, and bornyl acetate.

6. A method of elevating skin temperature, comprising applying a composition comprising an active ingredient to a subject, wherein the active ingredient comprises at least one of the members selected from the group consisting of an Arabian jasmine (matsurika, jasmin sambac) extract, a damask rose (Rosa damascena) steam distillate, 3-methyloctano-4-lactone, and bornyl acetate.

7. A method of preventing or improving shoulder stiffness or sensitivity to cold, comprising applying a composition comprising an active ingredient to a subject, wherein the active ingredient comprises at least one of the members selected from the group consisting of an Arabian jasmine (matsurika, jasmin sambac) extract, a damask rose (Rosa damascena) steam distillate, 3-methyloctano-4-lactone, and bornyl acetate.
